(19) United States
(12) Patent Application Publication
(10) Pub. No.: US 2023/0009906 A1
(43) Pub. Date: Jan. 12, 2023

(54) Tooth Whitening Kit and Method of Whitening Teeth

(71) Applicant: Colgate-Palmolive Company, New York, NY (US)

(72) Inventors: Leighton DAVIES-SMITH, Lebanon, NJ (US); Hallena STROTMAN, Somerset, NJ (US)

(73) Assignee: Colgate-Palmolive Company, New York, NY (US)

(21) Appl. No.: 17/370,358

(22) Filed: Jul. 8, 2021

(51) Int. Cl. A61C 19/06; A61N 5/06; A61C 17/00; A61K 8/22; A61Q 11/00

(52) U.S. Cl. CPC A61C 19/066; A61N 5/0603; A61N 5/062; A61C 17/00; A61K 8/22; A61Q 11/00; A61N 2005/0606; A61N 2005/0663

(57) ABSTRACT

A tooth whitening kit and method of whitening teeth using the same. The method may include applying a tooth whitening composition directly to surfaces of a user's teeth, exposing the surfaces of the user's teeth to ambient air for a first period of time to form a film of the tooth whitening composition on the surfaces of the user's teeth, applying light to the surfaces of the user's teeth for a second period of time while the film of the tooth whitening composition remains on the surfaces of the user's teeth, ceasing application of the light to the surfaces of the user's teeth and leaving the film of the tooth whitening composition on the surfaces of the user's teeth for a third period of time, and removing the film of the tooth whitening composition from the surfaces of the user's teeth.

### BACKGROUND

Tooth whitening is an increasingly popular treatment and dentists and patients alike are searching for techniques that are both convenient and comfortable while also being effective. Typically, to whiten a user's teeth a composition containing hydrogen peroxide is applied to the teeth and allowed to remain in contact with the teeth to be bleached for a period of time. Current systems are available that allow a user to apply radiation or light to the surfaces of the teeth that are pre-coated with the whitening composition to enhance the effectiveness of the whitening composition. However, due to legal requirements in some countries regarding the weight percentage of hydrogen peroxide permitted in tooth whitening compositions, the whitening results do not always meet the user's expectation. Thus, a need exists for a tooth whitening kit or method that meets legal requirements and consumer expectations.

### BRIEF SUMMARY

The present invention is directed to a tooth whitening kit and a method of whitening teeth using the same. The method may include applying a tooth whitening composition directly to surfaces of a user's teeth, exposing the surfaces of the user's teeth to ambient air for a first period of time to form a film of the tooth whitening composition on the surfaces of the user's teeth, applying light to the surfaces of the user's teeth for a second period of time while the film of the tooth whitening composition remains on the surfaces of the user's teeth, ceasing application of the light to the surfaces of the user's teeth and leaving the film of the tooth whitening composition on the surfaces of the user's teeth for a third period of time, and removing the film of the tooth whitening composition from the surfaces of the user's teeth.

In one aspect, the invention may be a method of whitening teeth comprising: applying a tooth whitening composition directly to surfaces of a user's teeth with a delivery device having a cavity that stores the tooth whitening composition; exposing the surfaces of the user's teeth with the tooth whitening composition thereon to ambient air for a first period of time to form a film of the tooth whitening composition on the surfaces of the user's teeth; after expiration of the first period of time, applying light to the surfaces of the user's teeth for a second period of time while the film of the tooth whitening composition remains on the surfaces of the user's teeth; after expiration of the second period of time, ceasing application of the light to the surfaces of the user's teeth and leaving the film of the tooth whitening composition on the surfaces of the user's teeth for a third period of time; and after expiration of the third period of time, removing the film of the tooth whitening composition from the surfaces of the user's teeth.

In another aspect, the invention may be a tooth whitening kit comprising: a delivery device comprising a cavity containing a tooth whitening composition, the tooth whitening composition comprising hydrogen peroxide, a polar solvent, a copolymer, and water, wherein the hydrogen peroxide is present in the tooth whitening composition in an amount of from about 0.1% to about 10% by weight; a tray device comprising an illumination source, the tray device comprising at least one channel configured to receive a plurality of a user's teeth and being configured to emit light onto facial surfaces of the plurality of the user's teeth; and a package containing the delivery device and the tray device.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 illustrates a kit including a package containing a delivery device and a tray device;

FIG. 2A is a front view of the delivery device of the kit of FIG. 1;

FIG. 2B is a cross-sectional area taken along line II-II of FIG. 2A;

FIG. 3A is a front view of the tray device of the kit of FIG. 1 in a non-activated state;

FIG. 3B is the front view of the tray device of FIG. 3B in an activated state such that the tray device is emitting light;

FIG. 4 is a flow chart describing a tooth whitening method utilizing the delivery device and the tray device of the kit of FIG. 1;

FIGS. 5A and 5B collectively illustrate a user actuating the delivery device to dispense a tooth whitening composition from a cavity thereof to an applicator thereof;

FIG. 6 illustrates the user applying the tooth whitening composition directly onto her teeth using the applicator of the delivery device;

FIG. 7 is a representation of a transformation of the tooth whitening composition after it is applied onto a user's tooth surface and exposed to ambient air to dry;

FIG. 8 illustrates the user with the tray device of the kit of FIG. 1 inserted into her mouth to emit light onto the teeth that are pre-coated with the tooth whitening composition;

FIG. 9 illustrates the user removing the tray device from her mouth;

FIG. 10 illustrates the user asleep with the tooth whitening composition remaining on her teeth;

FIG. 11 illustrates the user brushing her teeth to remove the tooth whitening composition therefrom;

FIG. 12 is a bar graph illustrating a comparison of the weight percentage of hydrogen peroxide in the tooth whitening composition when located in the delivery device and after application to the user's teeth;

FIG. 13 is a bar graph illustrating efficacy of various tooth whitening protocols;

FIG. 14 is a bar graph illustrating efficacy of a specific tooth whitening composition when used with and without light;

FIG. 15 is a bar graph illustrating efficacy of a specific tooth whitening composition when used with a light source over several treatments;

FIG. 16 is a bar graph illustrating efficiency of various tooth whitening protocols; and

FIG. 17 is a bar graph illustrating efficiency of various tooth whitening protocols.

### DETAILED DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

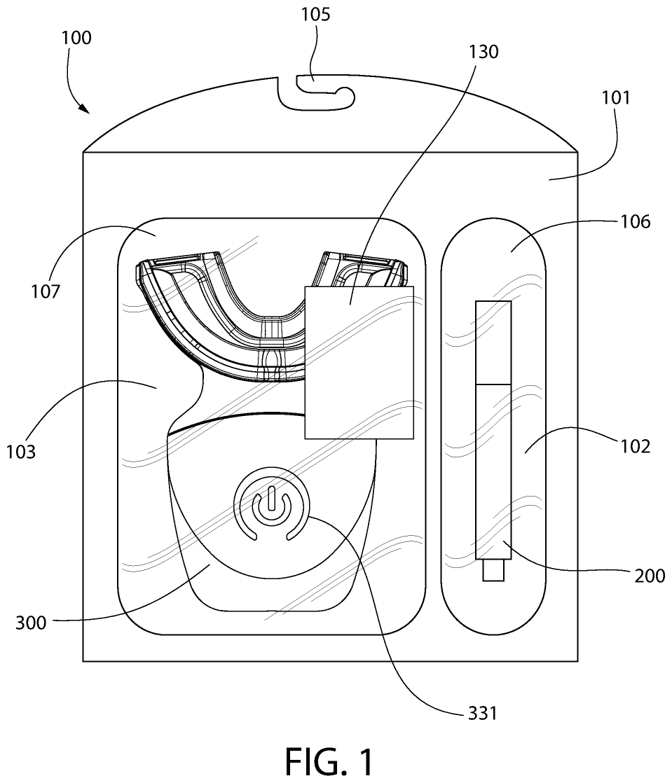

Referring first to FIG. 1, a kit 100 is illustrated in accordance with an embodiment of the present invention. The kit 100 is a tooth whitening kit including components that, when used together, provide optimal whitening to a user's teeth. The kit 100 is intended to be sold together and the parts contained within the kit 100, which will be described below, are to be used together to perform tooth whitening. The kit 100 may include an instruction sheet 130 which includes instructions regarding the use of the components of the kit 100 including order of application and use and timing sequences associated with the use of each product. Specifically, the instruction sheet (or instruction manual 130) comprises information and/or instructions sufficient to informal user how to properly use the components of the kit 100. The details about the method of use of the components of the kit 100 are provided in more detail below.

The kit 100 generally comprises a package 101, a delivery device 200, and a tray device 300. The package 101 may be any type of package commonly used for packaging merchandise at a point of sale. For example, the package 101 can be a clamshell package, a paperboard box, a sealed bag, a plastic container, a combination of plastic and paper, or the like. In the exemplified embodiment, the package 101 comprises a hanging element 105 that would allow for the package 101 to be hung from a post or the like as is common in retail locations. In other embodiments, the package 101 may not include such a hanging element, and may instead be stacked on a shelf or otherwise displayed. In the exemplified embodiment, the package 101 comprises a first compartment 102 within which the delivery device 200 is located and a second compartment 103 within which the tray device 300 is located. In other embodiments, the package 101 may define a singular compartment within which both the delivery device 200 and the tray device 300 are located. Furthermore, the package 101 includes a first window 106 that provides visual access into the first compartment 102 so that the delivery device 200 is visible within the first compartment 102 and a second window 107 that provides visual access into the second compartment 102 so that the tray device 300 is visible within the second compartment 103. In other embodiments, such as where the package 101 is a paperboard box, there may not be any windows and the delivery device 200 and the tray device 300 may not be visible until the package 101 is opened. The package 101 may include indicia including product and manufacturer information, images and photographs, and any other information desired to be displayed to a consumer.

The delivery device 200 is pre-loaded with a tooth whitening composition. Specifically, the delivery device 200 is filled with a tooth whitening composition prior to sale. The delivery device 200 may be refillable in some embodiments, or it may be disposable such that it is thrown away once its contents are fully dispensed. As discussed in greater detail below, after purchasing the kit 100, the user can use the delivery device 200 to apply the tooth whitening composition directly onto his or her teeth. The tray device 300 is configured to be inserted into the user's oral cavity or mouth so that the user's teeth rest within a channel of the tray device 300. The tray device 300 comprises an illumination source so that once activated the tray device 300 emits electromagnetic radiation (e.g., light) into the user's oral cavity and onto the surfaces of the teeth that have been pre-coated with the tooth whitening composition from the delivery device 200.

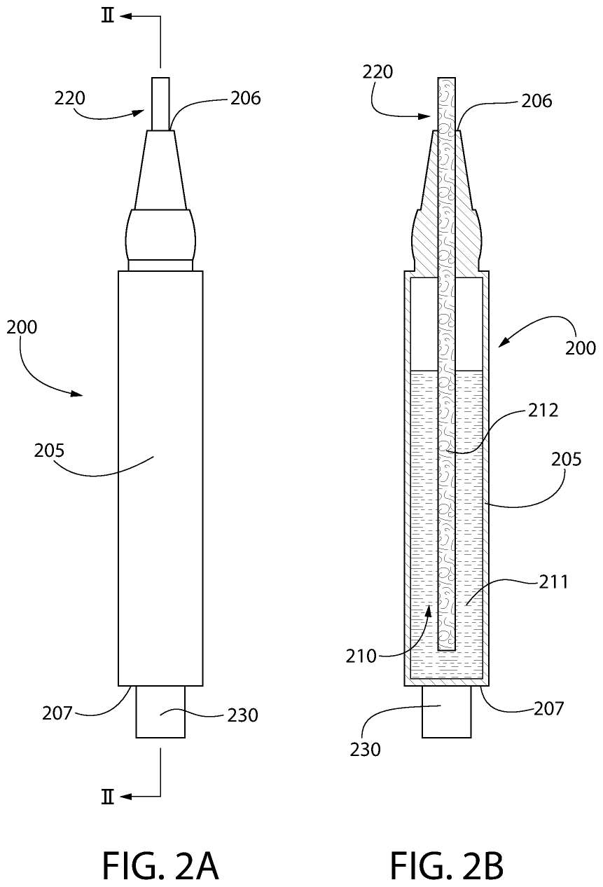

Referring to FIGS. 2A and 2B, the delivery device 200 will be described. The delivery device 200 is configured to contain a pre-loaded amount of the tooth whitening composition and to dispense the tooth whitening composition onto an applicator thereof for direct application onto facial surfaces of the user's teeth to whiten the teeth. In particular, the delivery device 200 comprises a housing 205 that defines a cavity 210 that contains the tooth whitening composition 211. Furthermore, the delivery device 200 comprises an applicator 220 protruding from a distal end 206 of the housing 205. The applicator 220 may take on many forms, such as being a plurality of bristles, a foam structure, a rubber element with or without protruding nubs, a capillary member, a structure with a flocked tip, or the like. In other embodiments, the delivery device 200 may comprise a stick or pole or the like that extends from the cap and has an applicator on its end. Thus, when the cap is removed from the housing, the applicator is also removed with the tooth composition thereon. The user can then apply the tooth whitening composition directly to the teeth using the applicator, before attaching the cap back onto the housing., In the exemplified embodiment, the tooth whitening composition 211 is a liquid. In other embodiments, the tooth whitening composition 211 may be a solid such that when dispensed the solid material protrudes from the distal end 206 of the housing 205 for direct application to a user's teeth (somewhat similar to deodorant application).

The delivery device 200 comprises an actuator 230 protruding from a proximal end 207 of the housing 205. In the exemplified embodiment, the actuator 230 is a depressible button-style device. That is, a user can press the actuator 230 in a direction towards the proximal end 207 of the housing 205, which will cause an amount of the tooth whitening composition to be dispensed from the cavity 210 to the applicator 220 that is protruding from the distal end 206 of the housing 205. The more times that the user presses the actuator 230, potentially the more volume of the tooth whitening composition 211 will be dispensed to the applicator 220. Thus, the user may be advised to only actuate the actuator 230 one time, or two to three times or the like before applying the tooth whitening composition 211 to the teeth in order to avoid coating the teeth with an excessive amount of the tooth whitening composition. This ensures that the tooth whitening composition coats only the teeth and not also the gums, and the user is able to easily control the application using the delivery device 200 and applicator 220 thereof.

The delivery device 200 may comprise any of various different dispensing mechanisms to achieve the dispensing of the tooth whitening composition therefrom. For example, the delivery device 200 may include an elevator mechanism that moves axially in a direction from the proximal end 207 towards the distal end 206 each time the actuator 230 is activated to dispense an amount of the tooth whitening composition 211. The delivery device 200 may include a rotary dial instead of a clickable actuator to achieve the movement of the elevator mechanism and dispensing of the tooth whitening composition. In other embodiments, such as the exemplified one, the delivery device 200 may include a delivery member 212 formed of a wicking material so that the tooth whitening composition 211 moves to the applicator 220 by capillary action. In such an embodiment, the actuator 230 may be able to be omitted. In still other embodiments, the housing 205 may be compressible/squeezable such that a user may squeeze the housing 205 to dispense the tooth whitening composition 211 from the cavity 210 to the applicator 220 (similar to dispensing toothpaste from a toothpaste tube). In still other embodiments, the delivery device 200 may operate much like a traditional soap dispenser such that it may include a pump and piston to facilitate dispensing of the tooth whitening composition 211. Thus, many techniques and components can be used for dispensing the tooth whitening composition 211 from the cavity 210 to the applicator 220 and the invention is not to be limited in this regard in all embodiments.

In the exemplified embodiment, the tooth whitening composition 211 may comprise hydrogen peroxide, a polar solvent (i.e., alcohol, which in one preferred embodiment is ethanol), a copolymer, and water. In some embodiments, the ethanol may have a weight percentage in a range of 60 wt % to 80 wt %, more specifically 70 wt % to 80 wt %, and still more specifically approximately 76 wt % of the tooth whitening composition 211. As described below, when the tooth whitening composition 211 is applied onto a user's teeth, the alcohol quickly evaporates. As a result, the weight percentage of the hydrogen peroxide in the tooth whitening composition 211 increases with the evaporation of the alcohol, thereby resulting in a more effective tooth whitening composition 211 in terms of tooth whitening efficacy (changing in color per treatment) and efficiency (change in color per minute of treatment time). This is an important feature of the tooth whitening composition 211 because some jurisdictions have legal requirements that limit the weight percentage of hydrogen peroxide that can be sold in a tooth whitening composition.

For example, in some countries, products with 0.1% hydrogen peroxide or less can be sold directly to consumers, whereas products with between 0.1% and 6% hydrogen peroxide can only be sold to dental practitioners (no retail sale directly to consumers). In other countries, for oral cosmetics containing more than 3% hydrogen peroxide, evidence that the product is safe for use when used as directed must be included. In still other countries, tooth whitening compositions may include no more than 6% hydrogen peroxide by weight. Other countries have still other regulations that must be complied with. Thus, the tooth whitening composition 211 sold in the kit 100 described herein may have a hydrogen peroxide weight percentage that falls within the law prior to use, but the weight percentage may increase once on the teeth and the alcohol evaporates. This allows for the better whitening effect from the higher weight percentage hydrogen peroxide to be obtained while meeting all legal requirements.

The tooth whitening composition may contain hydrogen peroxide in a range of from about 0.1% or about 10% by weight (the term “about” including plus or minus 5% of the value provided, as in 0.05 multiplied by the percentage listed so “about 10 wt %” includes a range of 9.5 wt % to 10.5 wt % and “about 0.1 wt %” includes a range of 0.095 wt % to 0.105 wt %). In some embodiments the hydrogen peroxide may be in a range of about 0.1 wt % to about 35 wt %. In some embodiments, the tooth whitening composition may contain hydrogen peroxide having a weight percentage of 0.1% or less, or in some embodiments about 0.1%. In other embodiments, the tooth whitening composition may contain hydrogen peroxide having a weight percentage of about 3%, or about 4.5%, or about 6%, or about 10%. In some embodiments, the copolymer of the tooth whitening composition may be a film forming hydrophobic polymer consisting essentially of 2-propenoic acid, 2-mthyl-, 2-methylproypl ester, polymer with 2-propenoic acid and N-(1,1,3,3-tetramethylbutyl)-2-propenamide, Arcylates/Dimethylaminoethyl Methacrylate copolymer, or mixtures thereof. The polar solvent may be an alcohol, and more specifically ethanol. Additional details about the tooth whitening composition 211 in accordance with one embodiment of the present invention are described in U.S. Pat. No. 10,821,066, the entirety of which is incorporated herein by reference.

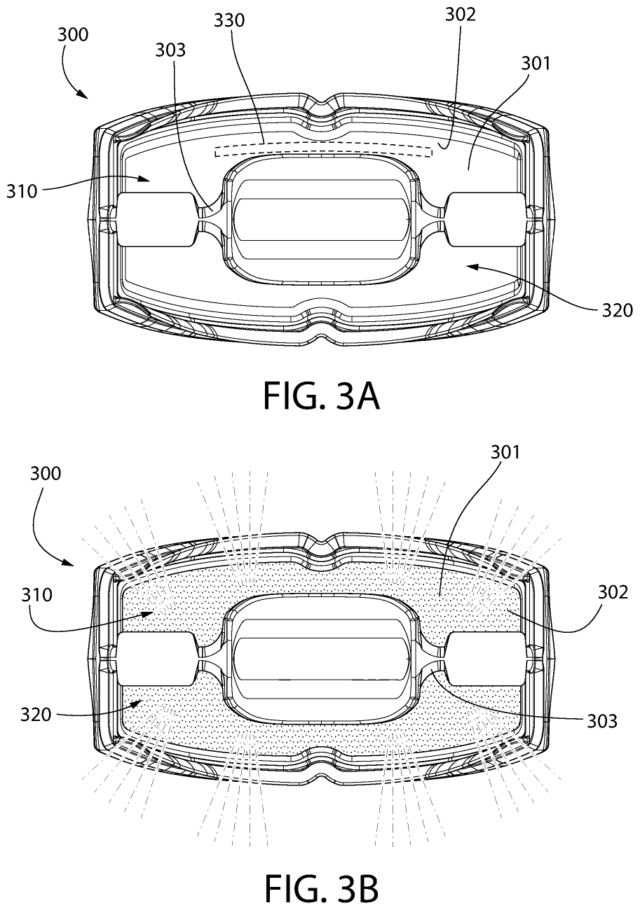

Referring to FIGS. 3A and 3B, the tray device 300 will be described. The tray device 300 comprises an arcuate rear wall 301 having a front surface 302 and a bite plate 303 protruding from the front surface 302 of the arcuate rear wall 301. The front surface 302 of the arcuate rear wall 301 is concave in the exemplified embodiment to allow for a good fit within a user's oral cavity. In the exemplified embodiment, the tray device 300 comprises a first channel 310 defined by an upper surface of the bite plate 303 and the arcuate rear wall 301 and a second channel 320 defined by a lower surface of the bite plate 303 and the arcuate rear wall 301. In other embodiments, the tray device 300 may include one of the first and second channels 310, 320, but not both. During use, the user's upper teeth are configured to nest within the first channel 310 while the user's lower teeth are configured to nest within the second channel 320. The facial surfaces of the user's teeth face the front surface 302 of the arcuate rear wall 301 when positioned in the first and second channels 310, 320.

The tray device 300 comprises an illumination source 330, which is illustrated generically as a dashed-line box in FIG. 3A. The illumination source 330 may be located within the housing of the tray device 300 behind the front surface 302 of the arcuate rear wall 301 such that once activated electromagnetic radiation is emitted through the front face 302 of the arcuate wall 301. The illumination source 330 may comprise light emitting diodes. For example, in one specific embodiment, the illumination source 330 may comprise light emitting diodes (LEDs) that are printed to a flexible lens plate. In other embodiments, the LEDs may be more conventional elements disposed within the tray device 300 and operably coupled to a power source. FIG. 3A illustrates the tray device 300 with the illumination source 330 in a non-activated state. FIG. 3B illustrates the tray device 300 with the illumination source 330 in an activated state. In this embodiment, the illumination source 330 comprises printed LEDs and there are possibly hundreds of distinct printed LEDs.

A user may activate the illumination source 330 by pressing a power button 331 (FIG. 1). The power button 331 may be a depressible button as shown in the exemplified embodiment, or it may be a slide switch, selector switch, joystick switch, pressure switch, temperature switch, or the like in other embodiments. In some embodiments upon pressing the power button 331 the illumination source 330 will alter into the activated state and remain in the activated state for a predetermined and pre-set period of time. In other embodiments, the illumination source 330 will remain in the activated state until the user presses the power button 331 a second time to deactivate the illumination source 330. In still other embodiments, the tray device 300 may be coupled to an electronic device such as a smart phone or a tablet or a computer via Bluetooth or other wireless technologies, or even through a wired attachment. The electronic device may have a software application that can control the activation and deactivation of the illumination source 330 wirelessly, rather than requiring the user to actuate a physical button.

When illuminated, the illumination source 330 may emit electromagnetic radiation or light at a wavelength of between 375 nm and 520 nm, more specifically between 380 nm and 430 nm, still more specifically between 390 nm and 420 nm, and still more specifically between 400 nm and 410 nm or 405 nm and 415 nm. In some specific embodiments, the wavelength is approximately 410 nm (approximately meaning a range of plus or minus 2 nm). In other embodiments, the wavelength is exactly 410 nm.

Additional details about one or more embodiments of the tray device 300 are disclosed in United States Patent Publication No. 2020/0114165, the entirety of which is incorporated herein by reference.

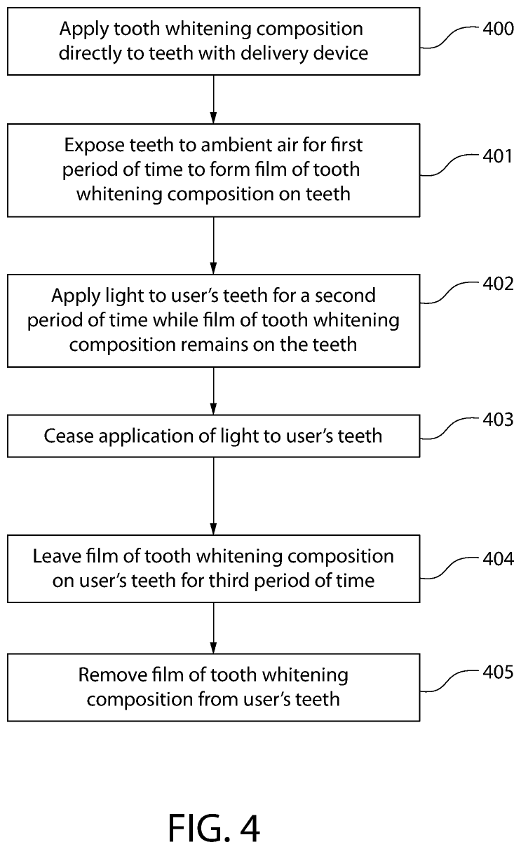

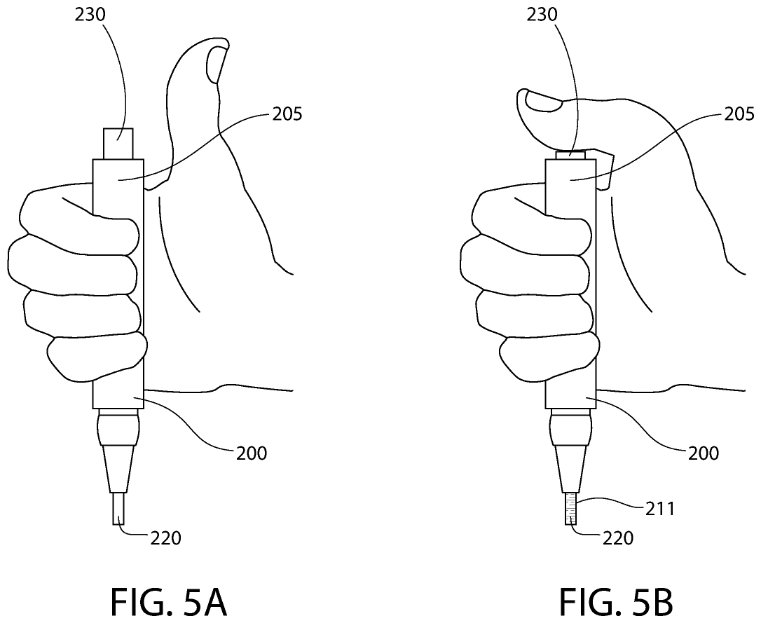

Referring to FIG. 4 in conjunction with FIGS. 5A-10, the method of whitening teeth using the delivery device 200 and the tray device 300 of the kit 100 will be described. First, referring to FIGS. 5A and 5B, the user will actuate the actuator 230 of the delivery device 200 to deliver an amount of the tooth whitening composition 211 contained in the cavity 210 of the delivery device 200 to the applicator 220 of the delivery device. FIG. 5B illustrates the tooth whitening composition 211 coating or located along the applicator 220. As noted above, in the exemplified embodiment the actuator 230 is actuated by a user pressing the actuator 230 towards the housing 210. However, other mechanisms are possible, including rotary knobs, slides, manual or electronic pumps, or the like.

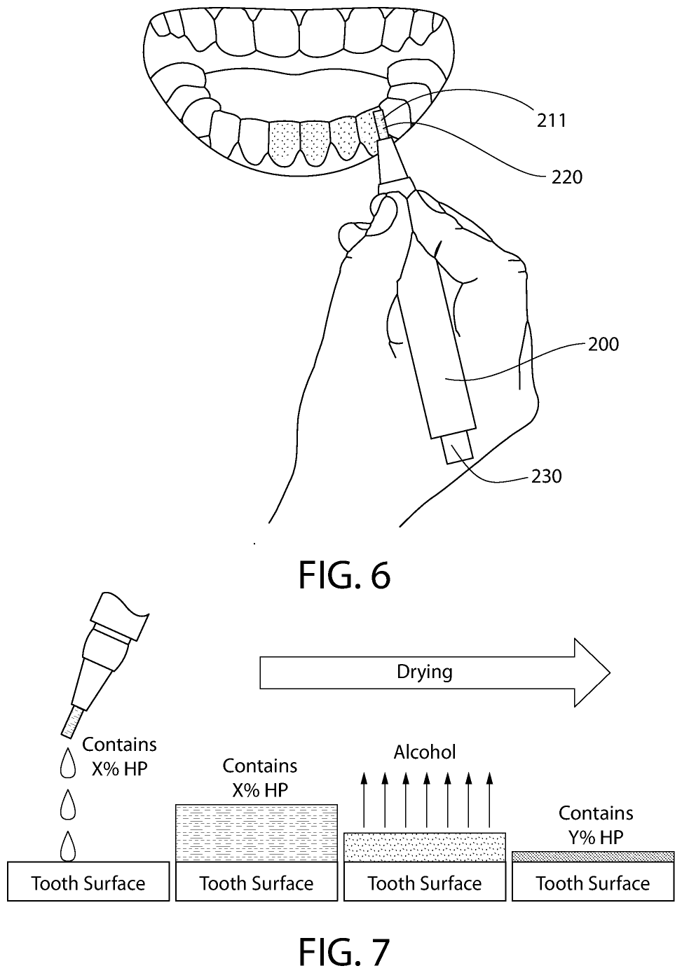

Once an amount of the tooth whitening composition 211 has been delivered to the applicator 220 of the delivery device 200, the user can apply the tooth whitening composition 211 from the applicator 220 of the delivery device 200 directly to the surfaces, preferably the facial surfaces, of the user's teeth. This is denoted as step 400 in FIG. 4 and is also illustrated in FIG. 6. Specifically, as shown in FIG. 6, the user will directly contact the applicator 220 of the delivery device 200 to the outer/facial surfaces of the teeth that the user desires to whiten. If necessary, the user can actuate the actuator 230 during this process if the user needs to replenish the tooth whitening composition 211 on the applicator 220 of the delivery device 200.

Once all of the teeth that the user desires to whiten have been coated with the tooth whitening composition 211, the user will allow the teeth coated with the tooth whitening composition 211 to remain exposed for a first period of time. This is denoted as step 401 in FIG. 4, and will be described with reference to FIG. 7. In particular, the user may leave his or her mouth partially open while pulling back his or her lips to expose the facial surfaces of the teeth that are coated with the tooth whitening composition for a sufficient amount of time to allow the tooth whitening composition to dry and form a film on the teeth. The film may be a transparent film, or a substantially transparent film such as one with at least 80% light transmissivity, or at least 90% light transmissivity in some embodiments.

As shown in FIG. 7, the tooth whitening composition containing X % hydrogen peroxide is applied onto the teeth. At this point, the hydrogen peroxide may have a weight percentage of 0.1%, or 3%, 6%, 10% or any weight percentage within the ranges described herein. Next, the user will expose the coated teeth to the ambient air by keeping the mouth open and/or the lips pulled back so that the teeth are exposed for the first period of time. During this first period of time, the alcohol in the tooth whitening composition will evaporate, leaving the remaining components of hydrogen peroxide, copolymer, and water in the film of the tooth whitening composition that is on the teeth. Due to the evaporation of the alcohol, the weight percentage of the remaining components in the film coated on the teeth increases. Thus, after expiration of the first period of time, the alcohol has fully evaporated and the film of the tooth whitening composition may include Y % hydrogen peroxide. The Y % may be at least two times the X % prior to the evaporation of the alcohol in some embodiments. The Y % may be at least three times the X % in other embodiments. In one particular embodiment, the X % may be 3% and the Y % may be approximately 12.5%. However, it should be appreciated that the weight percent of the hydrogen peroxide increases once the alcohol evaporates, thereby leaving a stronger tooth whitening composition on the teeth in the form of the film.

Once dried on the teeth and following the rapid evaporation of the alcohol, the tooth whitening composition polymer becomes a durable, non-smudge, peroxide-containing solid layer. Thus, even if the user closes his or her mouth at this point, the film of the tooth whitening composition will remain on the teeth. This is different from a traditional gel which would be removed from the teeth if rubbed against the inner lip after application to the teeth. Specifically, because the tooth whitening composition forms a non-smudge and durable film layer on the teeth, it remains so positioned until intentionally removed by the user, such as by the user brushing his or her teeth. The film is durable and can maintain extended contact with the teeth even for an overnight period of time (such as while the user sleeps) without disruption or dissolution, as the formed film is not soluble in water or saliva.

As noted above, the user should leave the teeth exposed for a first period of time to allow the alcohol to at least partially evaporate. The first period of time may be a length of time sufficient to allow the alcohol to fully evaporate in some embodiments, although this is not required in all embodiments. In some embodiments, the first period of time may be between 5 seconds and 30 seconds. In other embodiments, the first period of time may be between 5 seconds and 20 seconds. In still other embodiments, the first period of time may be at least 5 seconds, or at least 10 seconds, or at least 15 seconds, there being no maximum length of time. The first period of time is sufficient to enable the alcohol to dissolve, thereby leaving the film of the tooth whitening composition on the teeth with the higher weight percentage of hydrogen peroxide as compared to the weight percentage of the hydrogen peroxide stored in the cavity of the delivery device 200.

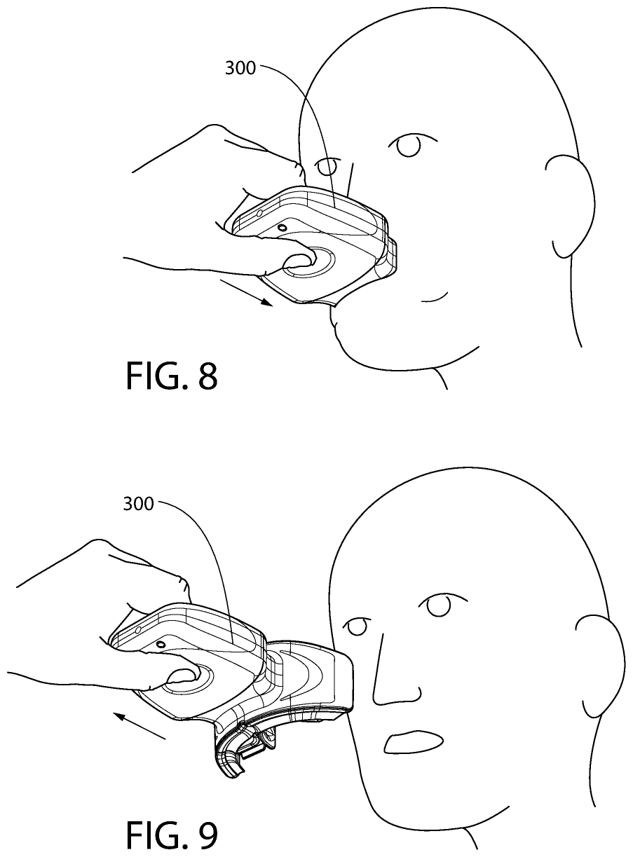

Next, as shown in step 402 of FIG. 4 and the accompanying illustration in FIG. 8, the tooth whitening method comprises applying electromagnetic radiation (e.g., light) to the surfaces of the user's teeth that are coated with the film of the tooth whitening composition. That is, the user leaves the film of the tooth whitening composition on the teeth after the alcohol has dissolved, and then applies electromagnetic radiation such as light to the coated teeth. The user may do this immediately after expiration of the first period of time, or may wait as long as the user chooses since the tooth whitening composition will not be removed from the teeth until intentionally removed by the user. In the exemplified embodiment, this step is achieved by the user inserting the tray device 300 into the oral cavity so that the upper teeth are located within the first channel 310 and the lower teeth are located within the second channel 320. The user can then actuate the power button 331, which in turn causes the illumination source to begin emitting electromagnetic radiation. As noted above, this may be achieved by LEDs in some embodiments. Moreover, the illumination source may emit electromagnetic radiation in a wavelength of any of the ranges noted above, including a range of 405 nm to 415 nm in some particular embodiments.

In accordance with the present invention, the electromagnetic radiation is to be applied onto the pre-coated surfaces of the user's teeth for a second period of time. As noted above, in some embodiments the tray device 300 may automatically deactivate the illumination source after expiration of the second period of time. In other embodiments, the tray device 300 may emit a sound, vibration, or light to inform the user of the expiration of the second period of time so that the user can manually power down the illumination source. In still other embodiments, the user may be required to manually track the time. In embodiments that have automatic operation, the tray device 300 may include a controller, a timer, and a power source that are operably coupled to the power button 331 and to the illumination source 330 (these features are described in US2020/0114165 which has been incorporated herein by reference above). The timer will track the time during which the illumination source 330 is activated, and the controller will automatically deactivate the illumination source upon expiration of the second period of time.

In some embodiments, the second period of time may be at least five minutes. In other embodiments, the second period of time may be between five minutes and thirty minutes. In still other embodiments, the second period of time may be between five minutes and twenty minutes, or between ten minutes and twenty minutes. Of course, other time ranges may be possible, including times longer than thirty minutes. However, for purposes of convenience it is generally desirable to keep treatment time to thirty minutes or less, and preferably twenty minutes or less.

It should be appreciated that the tooth whitening composition is not applied onto the first and second channels 310, 320 of the tray device 300 prior to inserting the tray device 300 into the user's mouth. Rather, the tooth whitening composition is only applied directly to the teeth, and not also to the tray. This is because the tooth whitening composition functions by allowing the alcohol to dissolve and leave a film on the teeth, so introducing the tooth whitening composition into the channels of the tray will not result in the most effective use of the tooth whitening composition. In fact, introducing the tooth whitening composition described herein into the tray device 300 will result in a film of the tooth whitening composition forming on the walls or floor of the tray device 300, which is not useful for whitening a user's teeth. Thus, in accordance with embodiments of the present invention, no tooth whitening composition should be dispensed onto the tray device (or light device) 300 prior to inserting the tray device 300 into the user's oral cavity.

Moreover, a user may elect to apply the tooth whitening composition onto only some, but not all, of the teeth. For example, the user may have one or two teeth that are a darker shade than the rest. In such a situation, the user may decide to apply the tooth whitening composition only onto those teeth that are darker than the others, in order to whiten them and make the shade consistent among all of the user's teeth. This cannot be achieved if the tooth whitening composition is introduced into a tray which is then inserted into the user's oral cavity.

Next, after expiration of the second period of time, at step 403 the application of light to the user's teeth ceases. As noted above, this may occur automatically by the tray device 300 or manually by the user. FIG. 9 illustrates the user removing the tray device 300 from his or her mouth/oral cavity at the end of the second period of time. As noted above, the tray device 300 may include an indicator, such as a sound, flash of light, vibration, or the like to indicate to the user that the second period of time has expired.

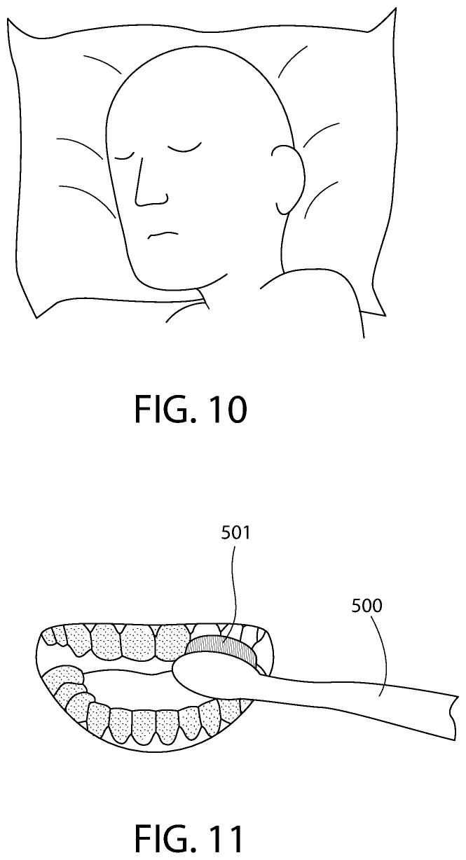

After expiration of the second period of time and removal of the tray device 300 from the user's oral cavity (or otherwise ceasing application of electromagnetic radiation or light to the user's oral cavity), the user will leave the film of the tooth whitening composition on his or her teeth, as set forth at step 404 of FIG. 4. The user should leave the film of the tooth whitening composition on his or her teeth for a third period of time. The third period of time may be at least thirty minutes in some embodiments. The third period of time may be between one hour and twelve hours in other embodiments. The third period of time may be between four hours and eight hours in other embodiments. In some embodiments, the user may be encouraged to go to sleep with the film of the tooth whitening composition still on his or her teeth, as shown in FIG. 10. This will ensure that the film of the tooth whitening composition remains on the teeth for the third period of time, which will be the amount of time that the user sleeps during an overnight sleep cycle (likely somewhere between six and eight hours).

After expiration of the third period of time, the user is encouraged to remove the film of the tooth whitening composition from his or her teeth, per step 405 shown in FIG. 4. In FIG. 11, the user is removing the tooth whitening composition from his or her teeth by brushing his or her teeth with a toothbrush 500. Specifically, the user is brushing his or her teeth with tooth cleaning elements or bristles 501 of the toothbrush 500. The user can also use mouthwash to rinse away any remaining amounts of the film on the teeth. Moreover, although in the exemplified embodiment the removal step requires an active step by the user such as brushing, the invention is not to be so limited in all embodiments. In other embodiments, the tooth whitening composition may be removed passively over a particular period of time, such as by dissolving or breaking apart within the user's mouth. For example, applying ethanol onto the teeth that are coated with the film of the tooth whitening composition results in the film of the tooth whitening composition being removed from the teeth.

The steps noted above may be included in text or images or photographs or a combination on the instruction sheet or manual 130. This will provide information to the user regarding how the products of the kit 100 should be used together in order to optimize tooth whitening results.

Experimental data has shown the following results. First, using the tooth whitening composition described herein with 3% hydrogen peroxide results in a color shade change of 3.5 shades in 21 days. Second, using the tooth whitening composition described herein with 3% hydrogen peroxide followed by a light treatment as described herein results in a color shade change of 6 shades in 10 days. Third, using the tooth whitening composition described herein with 6% hydrogen peroxide followed by a light treatment as described herein results in a color shade change of 6 shades in seven days and 7 shades in ten days. Finally, using the tooth whitening composition described herein with 10% hydrogen peroxide followed by a light treatment as described herein in three successive treatments back to back to back during a single treatment session results in a color shade change of 6 shades in 30 minutes. Thus, using this treatment option, a user can obtain optimal whitening in as little as 30 minutes of time. This last treatment option, which may be performed by a dental professional in some embodiments, will be described below.

If a user desires to obtain optimal results in the shortest possible period of time, the user may opt to use the last treatment option noted above with the 10% hydrogen peroxide. However, in some jurisdictions or countries such a treatment may be required to be performed by a dental professional. In other jurisdictions or countries, a user may be able to perform this treatment on his or her own. The treatment may be as follows. First, the user or a dental professional will apply the tooth whitening composition containing the 10 wt % hydrogen peroxide directly onto the teeth as described herein. The tooth whitening composition will be allowed to dry on the teeth by allowing the alcohol to dissolve to form the film as described herein above. Next, light will be applied onto the teeth that are pre-coated with the film of the tooth whitening composition for a specific period of time, which may be five minutes, or ten minutes, or fifteen minutes, or twenty minutes. In the exemplified embodiment, the light may be applied onto the teeth for ten minutes. Next, the user or the dental professional will wipe the teeth with ethanol (EtOH) to remove the film of the tooth whitening composition from the user's teeth. Specifically, the user or dental professional may wipe the teeth with a pad, such as a gauze pad or the like, which is coated or saturated with the ethanol to remove the tooth whitening composition from the teeth.

At some short interval after wiping the film of the tooth whitening composition off of the user's teeth (either immediately, or after a short break of one minute or two minutes or three minutes or four minutes or five minutes or the like), the user or dental professional will repeat the steps. Specifically, the user or dental professional will again coat the teeth with the tooth whitening composition containing 10 wt % hydrogen peroxide and ethanol and allow the tooth whitening composition to dry (by the evaporation of the ethanol, which may take five to twenty seconds as described herein). Once the tooth whitening composition has dried, light will be again applied to the teeth for the noted period of time (preferably about ten minutes). After expiration of the ten minutes, the tooth whitening composition will be removed from the user's teeth using ethanol.

After another short interval, these steps will be repeated one last time. Thus, the user's teeth will be again coated with the tooth whitening composition containing 10 wt % hydrogen peroxide and ethanol, which will then be permitted to dry. After drying, the light will be applied onto the teeth for the desired duration, preferably about ten minutes. Finally, after expiration of the desired duration, the tooth whitening composition will be removed from the teeth by wiping the teeth with ethanol (such as a gauze pad or the like coated or saturated with ethanol). Of course, after each treatment, the user may brush his or her teeth to remove the tooth whitening composition rather than wiping the teeth with ethanol.

The above describes a single treatment session that may take about 30 minutes, plus the amount of time it takes to coat the teeth with the tooth whitening composition and remove the film of the tooth whitening composition from the teeth in between applications. The thirty minute treatment session has been found to have whitening results that are as good as using a 35% hydrogen peroxide, with much less risk of damage to gums or pain to the user. Specifically, using a 35% hydrogen peroxide requires a long preparation time (up to an hour) so that the dental professional can protect the user's gums before applying the tooth whitening composition onto the teeth for safety reasons and to prevent damage to the gums and pain to the user. Thus, this 10 wt % hydrogen peroxide plus light application option, which has achieved proven results, is much more desirable than using a tooth whitening composition with a higher hydrogen peroxide content.

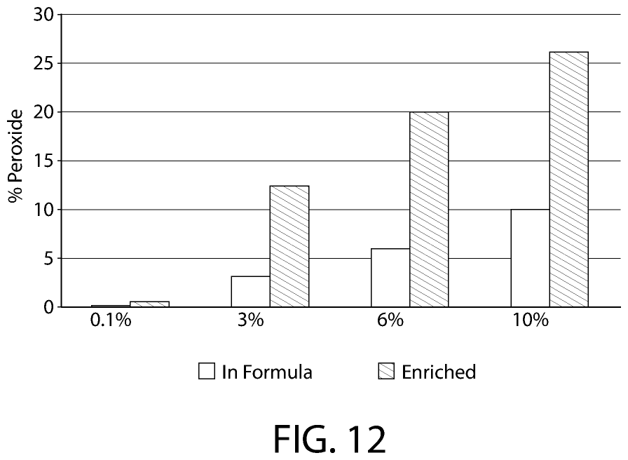

FIGS. 12-17 are graphical representations illustrating the effectiveness of using the tooth whitening composition described herein in conjunction with the tray device described herein and in the manner described herein and shown in FIG. 4.

FIG. 12 illustrates a weight percentage of hydrogen peroxide in the tooth whitening composition when it is located within the delivery device (In Formula) and when it is located on the teeth after the alcohol has dissolved (Enriched). As shown, when the hydrogen peroxide has a 0.1 wt % when in storage in the delivery device, once enriched due to dissolution of the alcohol the hydrogen peroxide is at least 0.2 wt %, or more specifically at least 0.3 wt %. When the hydrogen peroxide has a weight percentage of 3% when in storage in the delivery device, it may have a weight percentage of 12.5% when enriched. When the hydrogen peroxide has a weight percentage of 6% when in storage in the delivery device, it may have a weight percentage of 20% when enriched. When the hydrogen peroxide has a weight percentage of 10% when in storage in the delivery device, it may have a weight percentage of 26% when enriched. Thus, the weight percentage of the hydrogen peroxide doubles or triples or even more than triples in some instances once applied onto the teeth and allowed to sit for a sufficient amount of time for the alcohol to dissolve as described herein.

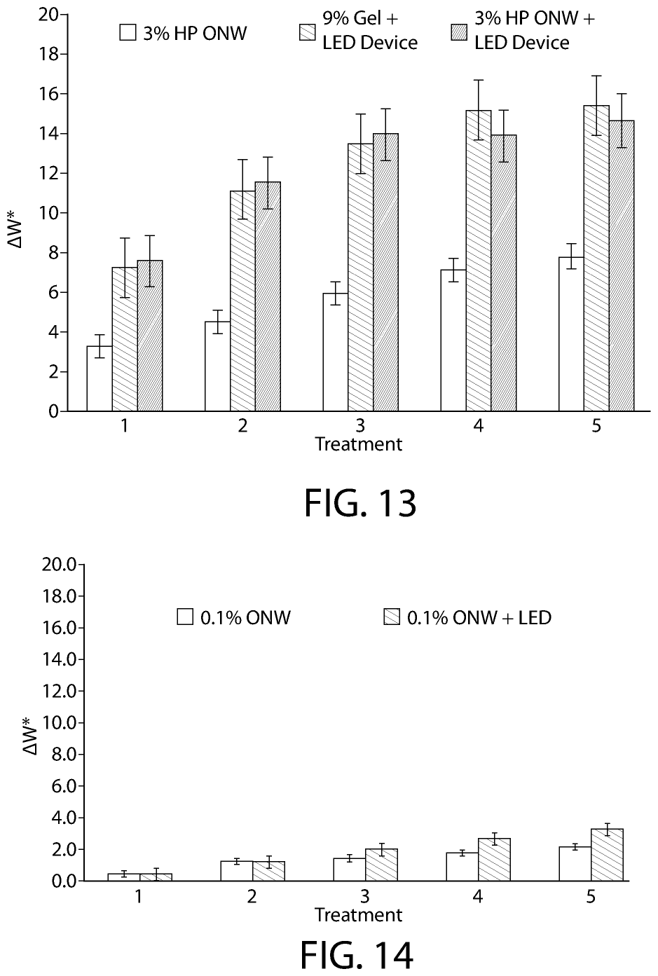

FIG. 13 is a bar graph illustrating a comparison of the whitening efficacy over a number of different treatments. Whitening efficacy is the change in whitening (AW) per treatment. The change in whitening may be measured on a tooth whitening chart or tooth shade guide (such as the Vita shade guides or the like). The bar graph of FIG. 13 compares whitening efficacy for a 3% hydrogen peroxide ONW tooth whitening composition (the letters ONW refer to the tooth whitening composition described herein that comprises hydrogen peroxide, a polar solvent, a copolymer, and water, and it is specifically a composition including hydrogen peroxide, alcohol, and a polymer/copolymer), a 3% hydrogen peroxide ONW tooth whitening composition plus a light device such as the tray device 300 described herein, and a 9% hydrogen peroxide gel (which does not include the alcohol that evaporates as with the ONW tooth whitening composition) plus a light device such as the tray device 300 described herein. That is, the gel tooth whitening composition is a more traditional tooth whitening composition that does not have evaporating alcohol as part of its formula and therefore the hydrogen peroxide weight percentage does not increase after it is applied onto the teeth. The FIG. 13 graph shows that the 9% gel plus LED device and the 3% ONW plus the LED device have very similar efficacy. In particular, the 3% ONW has a slightly greater efficacy over the first three treatments, and then the 9% gel has a slightly greater efficacy after treatments four and five. Both the 9% gel and the 3% ONW plus the light device have a better efficacy than the 3% ONW without the light device.

FIG. 14 is a bar graph that compares the tooth whitening efficacy for a 0.1% ONW tooth whitening composition both with and without a light device (such as the tray device 300 described herein). Again, a 0.1% ONW tooth whitening composition comprises 0.1 wt % hydrogen peroxide, plus a polar solvent, a copolymer, and water. The weight percentage of the hydrogen peroxide increases (possibly as much as three-fold as shown in FIG. 12) once applied onto the teeth and allowed to dry to form a film of the tooth whitening composition thereon. FIG. 14 illustrates that the whitening efficacy is better when the 0.1% ONW is used with a light device, particularly after treatment three, with the difference increasing for each treatment after the third treatment.

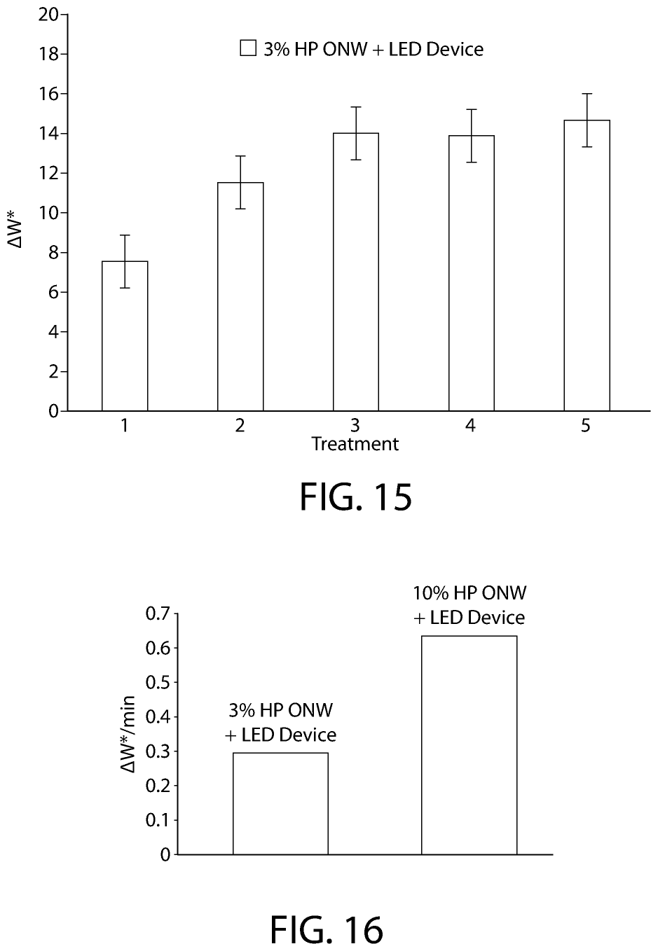

FIG. 15 is a bar graph illustrating the tooth whitening efficacy when using a 3% ONW tooth whitening composition plus a light device. As discussed previously, a 3% ONW tooth whitening composition comprises 3 wt % hydrogen peroxide, alcohol, copolymer, and water when in storage. The weight percentage of the hydrogen peroxide increases (as shown in the FIG. 12 bar graph) when the tooth whitening composition is applied to the teeth and allowed to dry such that the alcohol evaporates. It should be noted that the measurements in the graphs included herewith are performed on stained bovine teeth. Thus, in the experiments that achieved the data shown in the graphs, the delta W* whitening index is measured to reflect a change versus a baseline. When using bovine teeth, the whitening effectiveness plateaus around the fifth treatment, but this is not necessarily the case when using real human teeth.

FIG. 16 is a bar graph illustrating tooth whitening efficiency for different tooth whitening compositions and treatments. Tooth whitening efficiency is the change in whitening (AW) per minute of treatment time. Thus, if a treatment is ten minutes long, the total change in whitening divided by ten provides the tooth whitening efficiency. As shown, the tooth whitening composition with the higher percentage of hydrogen peroxide has the greatest tooth whitening efficiency.

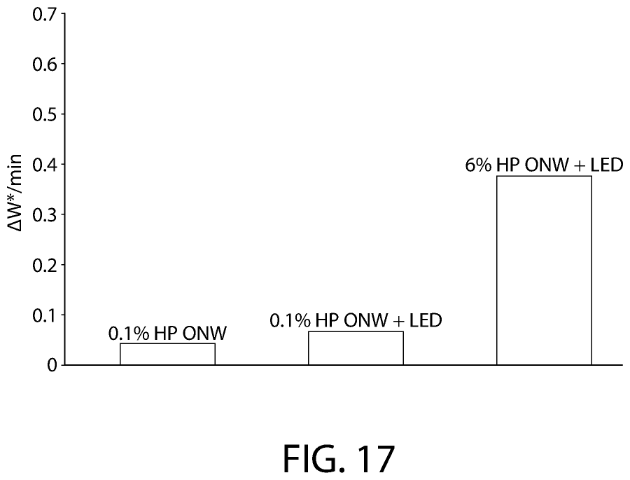

Finally, FIG. 17 is a bar graph illustrating tooth whitening efficiency for different tooth whitening compositions and treatments. FIG. 17 compares tooth whitening compositions including hydrogen peroxide, alcohol, copolymer, and water as described herein, except in the first two examples the tooth whitening composition has 0.1 wt % hydrogen peroxide and in the third example the tooth whitening composition has 6 wt % hydrogen peroxide. Furthermore, in the second and third examples a light device (i.e., the tray device 300) is used in the treatment, whereas no light is used in the first example. The results show a greater efficiency when a light device is used and when the hydrogen peroxide weight percentage is higher.

The use of a specific numerical value as a prefix for the various angles is not necessarily consistent in the specification and the claims. Rather, the numerical values used in the specification are merely used to distinguish one angle from another. The same is done in the claims, although the prefixes for the angles in the claims are simply listed sequentially as they appear in the claims rather than listed in a way that corresponds with the way that same angle is described in the specification.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

While the invention has been described with respect to specific examples including presently preferred modes of carrying out the invention, those skilled in the art will appreciate that there are numerous variations and permutations of the above described systems and techniques. It is to be understood that other embodiments may be utilized and structural and functional modifications may be made without departing from the scope of the present invention. Thus, the spirit and scope of the invention should be construed broadly as set forth in the appended claims.

1. A method of whitening teeth comprising:
applying a tooth whitening composition directly to surfaces of a user's teeth with a delivery device having a cavity that stores the tooth whitening composition;
exposing the surfaces of the user's teeth with the tooth whitening composition thereon to ambient air for a first period of time to form a film of the tooth whitening composition on the surfaces of the user's teeth;
after expiration of the first period of time, applying light to the surfaces of the user's teeth for a second period of time while the film of the tooth whitening composition remains on the surfaces of the user's teeth; and
after expiration of the second period of time, ceasing application of the light to the surfaces of the user's teeth and leaving the film of the tooth whitening composition on the surfaces of the user's teeth for a third period of time.

2. (canceled)

3. The method according to claim 1 wherein the first period of time is between five and twenty seconds, the second period of time is between five and thirty minutes, and the third period of time is between one and twelve hours.

4. The method according to claim 1 wherein the tooth whitening composition stored in the cavity of the delivery device comprises hydrogen peroxide at a first weight percentage, and wherein the film of the tooth whitening composition comprises the hydrogen peroxide at a second weight percentage that is greater than the first weight percentage.

5. The method according to claim 4 wherein the tooth whitening composition stored in the cavity of the delivery device comprises alcohol, and wherein as the tooth whitening composition dries on the surfaces of the user's teeth to form the film, the alcohol evaporates resulting in a weight percentage of the hydrogen peroxide in the tooth whitening composition increasing from the first weight percentage to the second weight percentage.

6. (canceled)

7. (canceled)

8. (canceled)

9. (canceled)

10. The method according to claim 1 wherein the tooth whitening composition comprises hydrogen peroxide, ethanol, a copolymer, and water, and wherein the ethanol rapidly evaporates when the surfaces of the user's teeth with the tooth whitening composition thereon are exposed to the ambient air for the first period of time, thereby increasing a weight percentage of the hydrogen peroxide.

11. The method according to claim 1 wherein applying light to the surface of the user's teeth comprises inserting a light device into the user's oral cavity, the light device emitting the light at a wavelength between 400 nm and 420 nm, and wherein the light device and the delivery device are packaged and sold together as a kit.

12. (canceled)

13. The method according to claim 11 wherein no tooth whitening composition is dispensed onto the light device prior to inserting the light device into the user's oral cavity.

14. The method according to claim 1 wherein the tooth whitening composition stored in the cavity of the delivery device comprises hydrogen peroxide in an amount of from about 0.1% by weight to about 10% by weight.

15. (canceled)

16. The method according to claim 1 further comprising, after expiration of the third period of time, removing the film of the tooth whitening composition from the surfaces of the user's teeth.

17. The method according to claim 16 wherein the removing the film of the tooth whitening composition from the surfaces of the user's teeth comprises brushing the user's teeth with tooth cleaning elements of a toothbrush.

18. A tooth whitening kit comprising:
a delivery device comprising a cavity containing a tooth whitening composition, the tooth whitening composition comprising hydrogen peroxide, a polar solvent, a copolymer, and water, wherein the hydrogen peroxide is present in the tooth whitening composition stored in the cavity of the delivery device in an amount of from about 0.1% to about 10% by weight;
a tray device comprising an illumination source, the tray device comprising at least one channel configured to receive a plurality of a user's teeth and being configured to emit light onto facial surfaces of the plurality of the user's teeth; and
a package containing the delivery device and the tray device.

19. The tooth whitening kit according to claim 18 wherein the tray device is configured to emit light at a wavelength between 400 nm and 420 nm, and wherein the delivery device comprises a body defining the cavity, an applicator, and an actuator, and wherein upon actuation of the actuator an amount of the tooth whitening composition is delivered from the cavity to the applicator for direct application onto a user's teeth.

20. The tooth whitening kit according to claim 18 wherein the copolymer is a film forming hydrophobic polymer consisting essentially of 2-propenoic acid, 2-mthyl-, 2-methylproypl ester, polymer with 2-propenoic acid and N-(1,1,3,3-tetramethylbutyl)-2-propenamide, Arcylates/Dimethylaminoethyl Methacrylate copolymer, or mixtures thereof, and wherein the polar solvent consists essentially of ethanol.

21. (canceled)

22. (canceled)

23. (canceled)

24. The tooth whitening kit according to claim 18 further comprising an instruction manual contained in the package, wherein the instruction manual comprises instructions for use of the delivery device and the tray device to whiten teeth, and wherein the instructions comprise information sufficient to instruct a user to perform the following steps:
dispense the tooth whitening composition from the cavity of the delivery device to an applicator of the delivery device and contact surfaces of the user's teeth with the applicator of the delivery device to apply the tooth whitening composition directly to the surfaces of the user's teeth;
expose the user's teeth with the tooth whitening composition thereon to ambient air for a first period of time to form a film of the tooth whitening composition on the surfaces of the user's teeth;
insert the tray device into an oral cavity of the user and activate the tray device so that the tray device emits the light onto the surfaces of the user's teeth that are coated with the film of the tooth whitening composition;
after expiration of a second period of time, remove the tray device from the oral cavity of the user; and
leave the tooth whitening composition on the user's teeth for a third period of time after removing the tray device from the oral cavity of the user.

25. (canceled)

26. A method of whitening teeth using the tooth whitening kit according to claim 18, the method comprising:
dispensing the tooth whitening composition from the cavity of the delivery device to an applicator of the delivery device and contacting surfaces of the user's teeth with the applicator of the delivery device to apply the tooth whitening composition directly to the surfaces of the user's teeth;
exposing the user's teeth with the tooth whitening composition thereon to ambient air for a first period of time to form a film of the tooth whitening composition on the surfaces of the user's teeth;
inserting the tray device into an oral cavity of the user and activating the tray device so that the tray device emits the light onto the surfaces of the user's teeth that are coated with the film of the tooth whitening composition;
after expiration of a second period of time, removing the tray device from the oral cavity of the user; and
leaving the tooth whitening composition on the user's teeth for a third period of time after removing the tray device from the oral cavity of the user.

27. The method according to claim 26 further comprising, after expiration of the third period of time, brushing the user's teeth to remove the tooth whitening composition therefrom, wherein the first period of time is at least five seconds, the second period of time is at least five minutes, and the third period of time is at least one hour.

28. (canceled)

29. A method of whitening teeth comprising:
a) applying a tooth whitening composition comprising ethanol and hydrogen peroxide directly to surfaces of a user's teeth and allowing the ethanol to evaporate thereby leaving a film of the tooth whitening composition on the surfaces of the user's teeth;
b) applying light to the surfaces of the user's teeth for a predetermined period of time while the film of the tooth whitening composition remains on the surfaces of the user's teeth;
c) after expiration of the predetermined period of time, ceasing application of the light to the surfaces of the user's teeth and removing the film of the tooth whitening composition from the surfaces of the user's teeth;
repeating steps a) through c) at least two times in succession within a single treatment session.

30. The method according to claim 29 further comprising repeating steps a) through c) at least three times in succession within a single treatment session.

31. (canceled)

32. The method according to claim 29 wherein the hydrogen peroxide is in an amount ranging from about 0.1 wt % to about 10 wt % and wherein the ethanol is in an amount ranging from about 70 wt % to about 80 wt %.

33. (canceled)

34. (canceled)

35. The method according to claim 29 wherein step c) comprises wiping the teeth with ethanol to remove the film of the tooth whitening composition from the surfaces of the user's teeth between each treatment.
